(19) United States
(12) Patent Application Publication
(10) Pub. No.: US 2009/0136667 A1
(43) Pub. Date: May 28, 2009

(54) NOVEL PORE-FORMING PRECURSORS COMPOSITION AND POROUS DIELECTRIC LAYERS OBTAINED THEREFROM

(76) Inventors: Joanne Deval, Paris (FR); Manon Vautier, Paris (FR)

Correspondence Address: AIR LIQUIDE;Intellectual Property, 2700 POST OAK BOULEVARD, SUITE 1800, HOUSTON, TX, 77056, US

(21) Appl. No.: 12/295,606

(22) PCT Filed: Mar. 20, 2007
(86) PCT No.: PCT/EP07/52661

§ 371 Date: Sep. 30, 2008

(30) Foreign Application Priority Data
Mar. 31, 2006 (FR) 0651126
Sep. 5, 2006 (FR) 0653576

(51) Int. Cl. C23C 16/00; C07D 493/08; C07C 47/225; C07D 303/04

(52) U.S. Cl. 427/255.23; 549/463; 549/397; 549/546; 568/425

(57) ABSTRACT

Method of forming a low dielectric k porous film on a substrate, comprising reacting at least a film matrix precursor compound having silicon, carbon, oxygen and hydrogen atoms, and either at least a pore-forming compound, of the formula (I) wherein R represents: either a linear or branched, saturated or non saturated hydrocarbon radical, or a cyclic saturated or unsaturated hydrocarbon radical, or at least one of the following pore-forming compounds: 1-methyl-4-(1-methyl ethyl)-7-oxabicyclo[2.2.1.]heptane, 1,3,3-trimethyl-2-oxabicyclo[2.2.1.]octane or 1,8-cineole, or 1-methyl-4-(1-methyl ethenyl)-7-oxabicyclo[4.1.0.]heptane; New precursor precursor mixture, and the use of a compound of formula (I), as a pore-forming compound in a chemical vapor deposition of a low dielectric k film on a substrate.

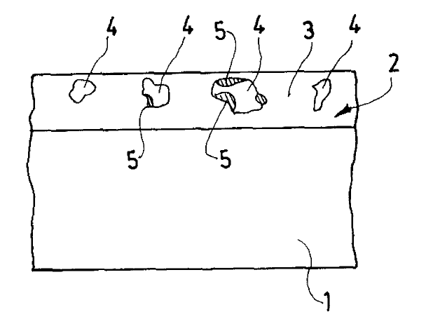

The present invention relates to pore-forming precursors which are able to generate matter-free volumes in a dielectric and also to the dielectric porous layers thus formed.

The insulating dielectric layers (also called “interlayer dielectrics”) used to separate metal interconnects between the various electrical circuits of an integrated circuit should have increasingly low dielectric constants.

For this, it is possible to create porosity in the dielectric itself (i.e. to create solid-matter-free micro-cavities) and thus to profit from the dielectric constant of air, which is equal to 1.

Reference is then made to ULK (or ultra-low dielectric constant or ultra-low-k) porous materials.

In order to produce such porous layers, conventional low dielectric constant precursors, also called matrix precursors, are associated, at the time of depositing, with organic compounds, which are organic pore-forming compounds which allow pores to be created in the “matrix” precursor.

The hybrid film, which is obtained for example by plasma enhanced chemical vapor deposition (PECVD) on a semiconductor substrate, is then subjected to a specific treatment (heating, exposure to ultraviolet radiation, electron bombardment), which results in the removal of a certain number of chemical molecules from the film (the organic molecules and/or their thermal decomposition products) and which creates solid-matter-free cavities in the “matrix” dielectric film (for example, an SiOCH film). For further details on the formation of these films reference may be made, for example, to international Application WO 2005/112095, or to United States Patent Application Nr. US-A-2002/037442 or to U.S. Pat. No. 6,312,793.

The objective of such films is to create a porosity in the dielectric matrix, without the structure of the film collapsing, i.e. to obtain a film that still has sufficient mechanical properties; the dielectric matrix is largely detailed in the herein above referenced patents or patent applications; it generally consists of a material deposited using precursor molecules containing silicon, carbon, oxygen and hydrogen atoms, more particularly siloxanes such as TMCTS (1,3,5,7-tetramethyl cyclotetrasiloxane), OMCTS (octamethyl cyclotetrasiloxane) or certain silane derivatives such as DEOMS (diethoxymethylsilane);

The latter step conditions the final success of the production of these films and the mechanical quality of the layers depends essentially on the choice of the combination of the matrix constitutive compounds and of the pore-forming compounds.

The hybrid material should preferably be at the same time, able to release matter under the effect of a treatment, to keep a stable framework during both this withdrawal step, and the subsequent semiconductor fabrication steps, in particular during the polishing steps of the dielectric layers.

The invention intends to solve the stated problem by virtue of the selection of suitable organic pore-forming compounds which, in combination with the matrix constitutive compounds, will generate a film on a substrate that has an ultra-low dielectric constant, while at the same time allowing the film to have a good mechanical strength.

The organic precursors according to the invention make it possible to solve the problem thus stated.

According to a first embodiment, the invention relates to a method of forming a low dielectric porous film on a substrate, comprising reacting at least a film matrix precursor compound having silicon, carbon, oxygen and hydrogen atoms, and either at least a pore-forming compound, of the formula (I):

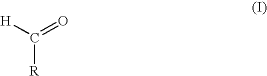

wherein R represents:

either a linear or branched, saturated or non saturated hydrocarbon radical, or a cyclic saturated or unsaturated hydrocarbon radical,

said cyclic or non cyclic radical being not substituted or substituted by one or more radicals selected from:

linear or branched alkyl radicals having from 1 to 4 carbon atoms;

linear or branched alkanoyl radicals having from 1 to 4 carbon atoms;

linear or branched alkanoyl oxy radicals having from 1 to 4 carbon atoms;

or at least one of the following pore-forming compounds:

1-methyl-4-(1-methyl ethyl)-7-oxabicyclo[2.2.1]heptane (more commonly known as 1,4-cineole) of the formula (II):

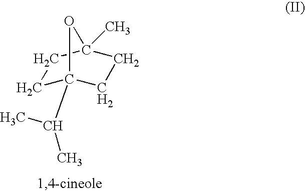

1,3,3-trimethyl-2-oxabicyclo[2.2.1]octane, or 1,8-cineole (or eucalyptol) of the formula:

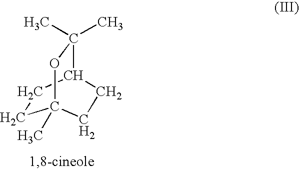

or 1-methyl-4-(1-methyl ethenyl)-7-oxabicyclo[4.1.0.]heptane, or limonene epoxide of the formula (IV):

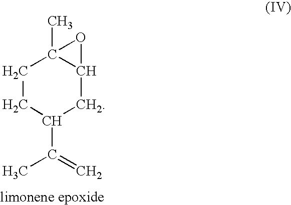

According to a more specific embodiment, the invention relates to a method as hereinbefore defined, wherein the pore-forming compound is a compound of the formula (Ia):

2,4-dimethyl-3-cyclohexene carboxaldehyde, or trivertal

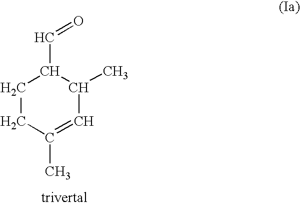

corresponding to the formula (I), wherein R represents a 2,4-dimethyl-3-cyclohexenyl radical, its positional and/or steric isomers and its derivatives, wherein one or more cyclic carbon atom is substituted by at least one alkyl radical having from one to six carbon atoms.

The porous layer of low dielectric constant k dielectric film obtained by the hereinabove defined from at least one film matrix precursor compound and at least one pore-forming compound as hereinbefore defined, is characterized in that it is composed of a plurality of first volumes comprising solid matter consisting of film matrix precursor compound and/or of derived matter, in particular derived subsequent to a heat treatment, of a plurality of second volumes not comprising solid matter and of a plurality of third volumes, generally arranged between at least one first and at least one second volume and representing less than 1% of the total volume of the porous layer, these third volumes consisting of at least one fraction of pore-forming compound and/or of derived matter, which may or may not be linked to the matrix precursor. The dielectric constant of said porous layer being less than or equal to 2.5.

The term “derived matter” is intended to mean the products derived from these precursors and which, subsequent to the treatment undergone by the layer, such as for example, heat treatment or ion bombardment, have been converted alone or on contact with the matrix molecules, so as to generate non-gaseous products which are incapable of being eliminated by diffusing through the layer, as the gaseous products derived from the decomposition of the organic precursors generally do.

According to a particular embodiment, the invention relates to a method as hereinbefore defined, wherein the said film matrix precursor compound is selected from siloxanes or silane derivatives and more particularly from TMCTS (1,3,5,7-tetramethyl cyclotetrasiloxane), OMCTS (octamethyl cyclotetrasiloxane) and DEOMS (diethoxymethylsilane);

This layer can be obtained by deposition on a substrate of the 300 mm wafer type in a “PECVD-type” reactor by injection of both the film matrix precursor compound and the pore-forming compound using a carrier gas, such as for example Helium, and then by heat treatment at a temperature below approximately 400° C.

The advantages of the pore-forming compounds according to the invention are the following:

Some of the molecules hereinabove mentioned are commercially available and relatively inexpensive; they have a moderate toxicity, a good volatility, and a reactive chemical function, for example, a carbon-carbon double bound, an epoxy function or a carbonyl function. They are generally chemically stable enough for packaging, transport and/or storage. and do not require the addition of a stabilizer.

However, it was observed that products which could be pore-forming compounds, such as, for example, alpha-terpinene or 1-isopropyl-4-methyl-1,3-cyclohexadiene, are not stable at the air exposure and suffer an oxidative degradation to produce some oxidized products, which could, in certain cases, also be pore-forming precursor materials for the production of low dielectric constant layers and that can also be used in the fabrication of semiconductors, while at the same time being stable to storage in the air and not liable to degrade.

One method of preparing these novel pore-forming compounds therefore consists, starting from alpha-terpinene or limonene, in oxidizing these products, preferably at a temperature above ambient temperature. Further details on such an oxidation is found, for example, in the article entitled “Thermal Degradation of Terrenes: Camphene's, Δ³-Careen, Limonene and α-Trepanned”; Environ. Sic. Techno.—1999, 33, 4029-4033 or in the article entitled “Determination of Limonene Oxidation Products using SPUME and GC-MS”, Journal of Chromatographic Science, Vol. 41, January 2003.

In particular, it has been demonstrated that, starting from the oxidation of alpha-trepanned, it is possible to generate:

1,4-cineole, or 1-(1-methyl ethyl)-4-methyl-7-oxabicyclo[2.2.1.]heptane, a molecule of low toxicity;

1,8-cineole, or eucalyptol, or else 1,3,3-tri-methyl-2-oxabicyclo[2.2.2.]octane, a molecule which is itself also of very low toxicity

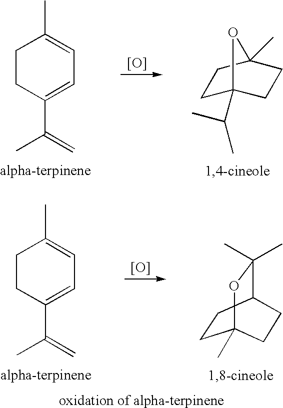

Similarly, starting from limonene, it is possible to generate:

limonene oxide or 4-isopropenyl-1-methyl-1-cyclo-hexene-1,2-epoxide:

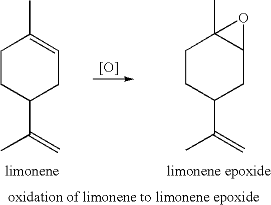

Trivertal or 2,4-dimethyl-3-cyclohexane, is a commercially available product, and is already in an oxidized state

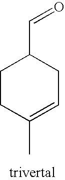

The single FIGURE schematically shows the porous layer obtained according to the invention: A layer 2 was deposited, on a substrate 1, by the “PECVD” process, said layer consisting of a mixture of a “matrix” precursor 3 and of an organic precursor deposited using their gaseous phases. The whole is subsequently subjected, in a manner known per se, to a heat treatment step, at a temperature of the order of approximately 300° C. to 400° C., generally lasting several tens of minutes, possibly followed by an ion bombardment step, then optionally by a treatment in a moist atmosphere and they drying, as described, for example, in US-A-2005/0227502. In the course of the heat treatment, the organic precursor is decomposed under the effect of the heat, giving rise to matter-free cavities 4, with, however, a few volumes 5 in which it is possible to identify residual organic matter that has not been completely decomposed, these volumes 5 being located between the matrix precursor volume 3 and the matter-free volumes 4. These volumes 5 will preferably always represent less than 1 vol % of the layer after thermal (or other) treatment, more preferably less than a few hundred ppm. The matrix precursor volume 3 (also called first volume in the present application) generally consists of a single volume exhibiting continuity (giving the layer the desired mechanical strength), in which are located a plurality of second and third volumes 4 and 5.

According to another embodiment the invention relates to a precursor mixture comprising at least a film matrix precursor compound having silicon, carbon, oxygen and hydrogen atoms, and either at least a pore-forming compound, of the formula (I):

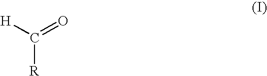

wherein R represents:

either a linear or branched, saturated or non saturated hydrocarbon radical, or a cyclic saturated or unsaturated hydrocarbon radical,

said cyclic or non cyclic radical being not substituted or substituted by one or more radicals selected from:

linear or branched alkyl radicals having from 1 to 4 carbon atoms;

linear or branched alkanoyl radicals having from 1 to 4 carbon atoms;

linear or branched alkanoyl oxy radicals having from I to 4 carbon atoms;

or at least one of the following pore-forming compounds:

1-methyl-4-(1-methyl ethyl)-7-oxabicyclo[2.2.1.]heptane of the formula (II):

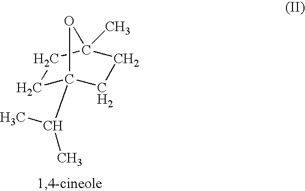

1,3,3-trimethyl-2-oxabicyclo[2.2.1.]octane, or 1,8-cineole (or eucalyptol) of the formula:

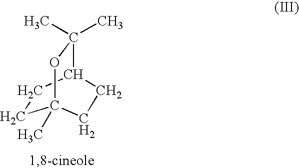

or 1-methyl-4-(1-methyl ethenyl)-7-oxabicyclo[4.1.0.]heptane or limonene epoxide of the formula (IV):

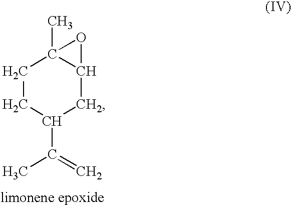

and more specifically to a precursor mixture as hereinabove defined, wherein the pore-forming compound is a compound of the formula (Ia):

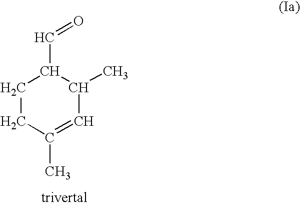

corresponding to the formula (I), wherein R represents a 2,4-dimethyl-3-cyclohexenyl radical.

According to another embodiment, the invention relates to the use of a compound of the formula (I):

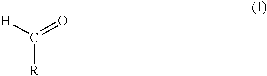

wherein R represents:

either a linear or branched, saturated or non saturated hydrocarbon radical, or a cyclic saturated or unsaturated hydrocarbon radical,

said cyclic or non cyclic radical being not substituted or substituted by one or more radicals selected from:

linear or branched alkyl radicals having from 1 to 4 carbon atoms;

linear or branched alkanoyl radicals having from 1 to 4 carbon atoms;

linear or branched alkanoyl oxy radicals having from 1 to 4 carbon atoms;

or of the following compounds:

1-methyl-4-(1-methyl ethyl)-7-oxabicyclo[2.2.1.]heptane of the formula (II):

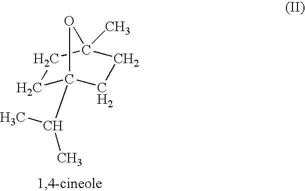

1,3,3-trimethyl-2-oxabicyclo[2.2.1.]octane, or 1,8-cineole (or eucalyptol) of the formula:

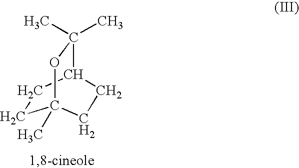

or 1-methyl-4-(1-methyl ethenyl)-7-oxabicyclo[4.1.0.]heptane or limonene epoxide of the formula (IV):

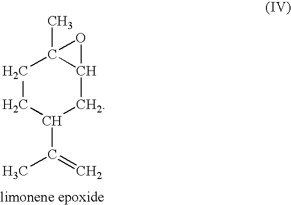

as a pore-forming compound in a chemical vapor deposition of a low dielectric k film on a substrate.

These porous layers which have a low dielectric constant usually less than 2.5 can be used in the fabrication of integrated circuits, flat screens, memories (in particular “random access” memories) and in any similar applications in which a low dielectric constant dielectric layer is used to isolate two electrical components (dielectric interconnection layers). They will more particularly be used in the circuits for interconnecting the various components of an integrated circuit, called BEOL (“Back end of the line”).

Porous low k films have been obtained using the following process and conditions:

The deposits were performed on a 6″ plasma enhanced chemical vapor deposition (PECVD) reactor. Hybrid films obtained were then annealed in a tube furnace at temperatures between 400° C. to 470° C. for 15 to 60 minutes under N2 flow, with additives such as H2 or O2 at concentrations between 1% and 20%.

Thickness and refractive index were measured on a Filmmetrics reflectometer. Dielectric constants were determined using a MDC mercury probe with a HP capacimeter.

Deposition was performed at pressures between 0.5 and 2 Torr, with radio-frequency power between 100 W and 250 W at 13.56 MHz, by co-depositing a Si-based precursor (diethoxymethylsilane) with described pore-forming compounds (Trivertal) onto a silicon wafer.

Flow rates of diethoxymethylsilane and pore-forming compound were varying in the range 125-500 mg/min (TEOS equivalent on a thermal mass-flow meter). Helium was used at 500 sccm as carrier gas. Deposition times ranges between 30 s and 7 min. Thickness between 100 nm and 700 nm was obtained. After annealing, thickness between 100 and 600 nm was obtained. Refractive index between 1.29 and 1.35 was obtained, and k value between 2.1 and 2.5

1-7. (canceled)

8. A method of forming a low dielectric k porous film on a substrate, comprising reacting at least a film matrix precursor compound having silicon, carbon, oxygen and hydrogen atoms, and either at least a pore-forming compound, of the formula (I): 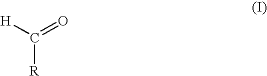
wherein R represents:
either a linear or branched, saturated or non saturated hydrocarbon radical, or a cyclic saturated or unsaturated hydrocarbon radical,
said cyclic or non cyclic radical being not substituted or substituted by one or more radicals selected from:
linear or branched alkyl radicals having from 1 to 4 carbon atoms;
linear or branched alkanoyl radicals having from 1 to 4 carbon atoms;
linear or branched alkanoyl oxy radicals having from 1 to 4 carbon atoms;
or at least one of the following pore-forming compounds:
1-methyl-4-(1-methyl ethyl)-7-oxabicyclo[2.2.1.]heptane of the formula (II): 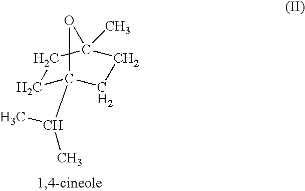
1,3,3-trimethyl-2-oxabicyclo[2.2.1.]octane or 1,8-cineole (or eucalyptol) of the formula: 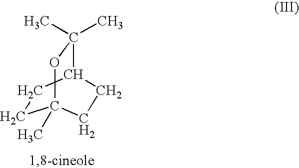
or 1 -methyl-4-(1 -methyl ethenyl)-7-oxabicyclo[4.1.0.]heptane or limonene epoxide of the formula (IV):

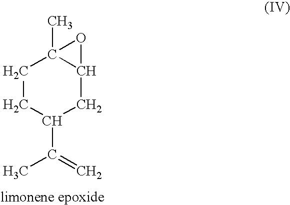

9. The method of claim 8, wherein the pore-forming compound is a compound of the formula (Ia): 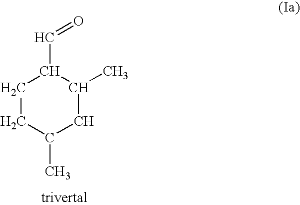
corresponding to the formula (I), wherein R represents a 2,4-dimethyl-3-cyclohexenyl radical.

10. The method of claim 8, wherein the said film matrix precursor compound is selected from siloxanes or silane derivatives.

11. The method of claim 10, wherein the said film matrix precursor compound is selected from TMCTS (1,3,5,7-tetramethyl cyclotetrasiloxane), OMCTS (octamethyl cyclotetrasiloxane) and DEOMS (diethoxymethylsilane).

12. A precursor mixture comprising at least a film matrix precursor compound having silicon, carbon, oxygen and hydrogen atoms, and either at least a pore-forming compound, of the formula (I): 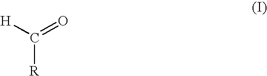
wherein R represents:
either a linear or branched, saturated or non saturated hydrocarbon radical, or a cyclic saturated or unsaturated hydrocarbon radical,
said cyclic or non cyclic radical being not substituted or substituted by one or more radicals selected from:
linear or branched alkyl radicals having from 1 to 4 carbon atoms;
linear or branched alkanoyl radicals having from 1 to 4 carbon atoms;
linear or branched alkanoyl oxy radicals having from 1 to 4 carbon atoms;
or at least one of the following pore-forming compounds:
1-methyl-4-(1-methyl ethyl)-7-oxabicyclo[2.2.1 ]heptane of the formula (II): 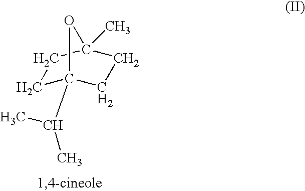
1,3,3-trimethyl-2-oxabicyclo[2.2.1]octane or 1,8-cineole (or eucalyptol) of the formula: 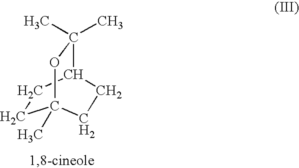
or 1-methyl-4-(1-methyl ethenyl)-7-oxabicyclo[4.1.0]heptane or limonene epoxide of the formula (IV):

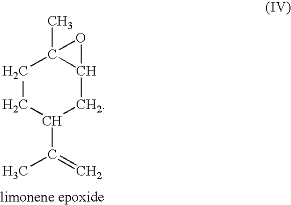

13. The precursor mixture of claim 12, wherein the pore-forming compound is a compound of the formula (Ia): 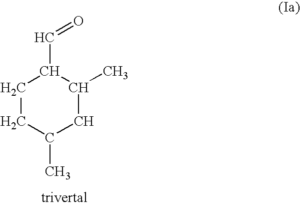
corresponding to the formula (I), wherein R represents a 2,4-dimethyl-3-cyclohexenyl radical.

14. Use of a compound of the formula (I): 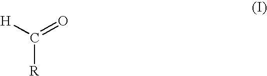
wherein R represents:
either a linear or branched, saturated or non saturated hydrocarbon radical, or a cyclic saturated or non saturated hydrocarbon radical,
said cyclic or non cyclic radical being not substituted or substituted by one or more radicals selected from:
linear or branched alkyl radicals having from 1 to 4 carbon atoms;
linear or branched alkanoyl radicals having from 1 to 4 carbon atoms;
linear or branched alkanoyl oxy radicals having from 1 to 4 carbon atoms;
or of the following compounds:
1-methyl-4-(1-methyl ethyl)-7-oxabicyclo[2.2.1 ]heptane of the formula (II): 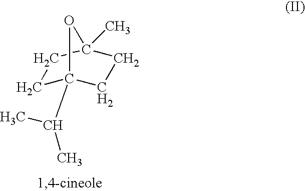
1,3,3-trimethyl-2-oxabicyclo[2.2.1]octane, or 1,8-cineole (or eucalyptol) of the formula: 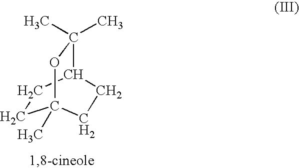
or 1-methyl-4-(1-methyl ethenyl)-7-oxabicyclo[4.1.0]heptane or limonene epoxide of the formula (IV): 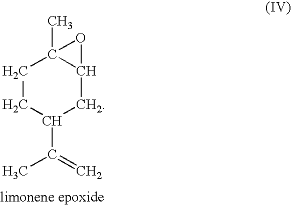
as a pore-forming compound in a chemical vapor deposition of a low dielectric k film on a substrate.
